(19) United States
(12) Patent Application Publication
(10) Pub. No.: US 2025/0258169 A1
(43) Pub. Date: Aug. 14, 2025

(54) SUPPORT, LIGAND-BONDED SUPPORT, AFFINITY PARTICLE, REAGENT AND KIT INCLUDING THE SAME, AND DETECTION METHOD

(71) Applicant: CANON KABUSHIKI KAISHA, Tokyo (JP)

(72) Inventors: RYO NATORI, Tokyo (JP); YUSUKE KOSAKI, Kanagawa (JP); KAZUNORI NOGUCHI, Ibaraki (JP); MASASHI KAWAMURA, Kanagawa (JP); HIDEHARU SHIMOZAWA, Tokyo (JP); YUNA YAMAMOTO, Tokyo (JP); SHUHEI ISHIOKA, Kanagawa (JP)

(21) Appl. No.: 19/045,692

(22) Filed: Feb. 5, 2025

(30) Foreign Application Priority Data
Feb. 13, 2024 (JP) 2024-019747

(51) Int. Cl. G01N 33/547; C07C 321/14; C07D 303/24; G01N 33/543; G01N 33/68

(52) U.S. Cl. CPC G01N 33/547; C07C 321/14; C07D 303/24; G01N 33/54393; G01N 33/6872; G01N 2333/4737

(57) ABSTRACT

A support including: a substrate; and a structure having a reactive functional group bonded to the substrate through a structure represented by the formula (1), wherein the structure having a reactive functional group has as the reactive functional group at least one selected from the group consisting of: a carboxy group; a maleimide group; a carbamoyl group; a tosyl group; an amino group; an epoxy group; and a thiol group:

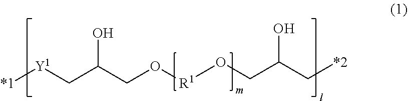

in the formula (1), “1” represents an integer of 1 or more and 11 or less, “m” represents an integer of 1 or more and 10 or less, Y¹ represents a sulfur atom, a structure represented by the formula (2), or a structure represented by the formula (3), and R¹ represents an unsubstituted alkylene group having 2 to 4 carbon atoms.

### BACKGROUND OF THE INVENTION

### Field of the Invention

The present invention relates to a support, a ligand-bonded support, an affinity particle, a reagent and a kit each including the same, and a detection method.

### Description of the Related Art

A method utilizing a solid-phase support in in vitro diagnostics for use in diagnosis of diseases has been known as a method of detecting a target substance. This method is a method in which a ligand is immobilized to a solid-phase support and brought into contact with a sample to cause a target substance to react with the ligand.

In this method, during the above-mentioned contact, the target substance and impurities in the sample each nonspecifically adsorb to the surface of the solid-phase support, but not the ligand, and the adsorption becomes noise in some cases. Accordingly, in the method utilizing the solid-phase support, it is desired that the solid-phase support have a low adsorbing property to a substance other than the target substance, which is called nonspecific adsorption.

There is a method of coating the surface of the solid-phase support with a biologically derived macromolecule, such as albumin, casein, or gelatin, at a downstream process as a method of reducing the nonspecific adsorption. However, those biologically derived substances have different physical properties for production lots in some cases.

Thus, when the surface of the solid-phase support is coated at the downstream process, a method using a hydrophilic macromolecule having high biocompatibility instead of the biologically derived macromolecule is also effective as the method of reducing the nonspecific adsorption. However, when the adsorption of the macromolecules to the solid-phase support is based on physical adsorption, the macromolecules may be liberated by dilution, and the nonspecific adsorption cannot be sufficiently suppressed in some cases.

A technology including introducing a glycidyl group into the surface of the solid-phase support and modifying the glycidyl group to suppress nonspecific adsorption has been known as a method of suppressing liberation of a nonspecific adsorption-suppressing component on the surface of the solid-phase support.

In each of Japanese Patent Application Laid-Open No. 2000-351814 and International Publication No. WO2007/063616, there is a disclosure of a solid-phase support obtained by chemically bonding a ligand to a particle in which polyglycidyl methacrylate is arranged on its surface.

The support in the related art has room for improvement in terms of nonspecific adsorption.

### SUMMARY OF THE INVENTION

An object of the present invention is to provide a support that has low nonspecific adsorption and is capable of detecting a target substance having a low concentration (i.e., has high sensitivity), a ligand-bonded support, an affinity particle, a kit and a reagent each including the same, and a detection method.

The present invention relates to a support including: a substrate; and a structure having a reactive functional group bonded to the substrate through a structure represented by the following formula (1), wherein the structure having a reactive functional group has as the reactive functional group at least one selected from the group consisting of: a carboxy group; a maleimide group; a carbamoyl group; a tosyl group; an amino group; an epoxy group; and a thiol group:

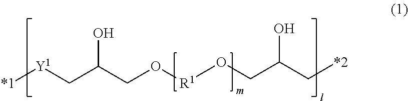

in the formula (1), “1” represents an integer of 1 or more and 11 or less, “m” represents an integer of 1 or more and 10 or less, Y¹ represents a sulfur atom, a structure represented by the following formula (2), or a structure represented by the following formula (3), R¹ represents an unsubstituted alkylene group having 2 to 4 carbon atoms, *1 represents a bonding position to a carbon atom or a sulfur atom in the substrate or the structure having a reactive functional group, *2 represents a bonding position to a nitrogen atom, a sulfur atom, or an oxygen atom in the substrate or the structure having a reactive functional group, provided that when *1 represents a bonding position to an atom in the substrate, *2 represents a bonding position to an atom in the structure having a reactive functional group, and when *1 represents a bonding position to an atom in the structure having a reactive functional group, *2 represents a bonding position to an atom in the substrate;

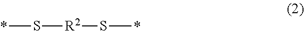

in the formula (2), R² represents an alkylene group having 1 to 8 carbon atoms, and the alkylene group represented by R² may have at least one of a halogen atom and a hydroxy group as a substituent, and * represents a bonding position in the structure represented by the formula (1);

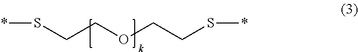

in the formula (3), “k” represents an integer of 1 or more and 8 or less, and * represents a bonding position in the structure represented by the formula (1).

The present invention also relates to a ligand-bonded support in which a ligand is chemically bonded to the above-mentioned support through a reactive functional group.

The present invention also provides an affinity particle in which a ligand is chemically bonded to the above-mentioned support through a reactive functional group, and a reagent for use in detection of a target substance in a specimen by in vitro diagnosis, the reagent including the affinity particle. The present invention also provides a kit for use in detection of a target substance in a specimen by in vitro diagnosis, the kit including a cartridge including the above-mentioned affinity particle. The present invention also relates to a method of detecting a target substance in a specimen by in vitro diagnosis, the method including mixing the above-mentioned reagent and a specimen that may contain the target substance.

The present invention also provides a compound represented by the following formula (5-1).

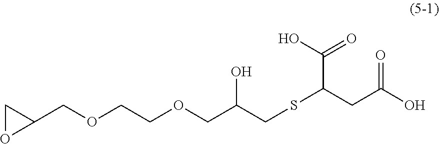 [SMILES: O=C(O)CC(SCC(O)COCCOCC1CO1)C(=O)O]

In addition, the present invention provides a compound represented by the following formula (5-2).

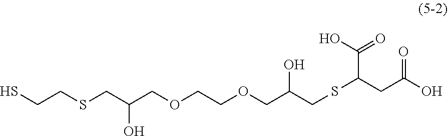 [SMILES: O=C(O)CC(SCC(O)COCCOCC(O)CSCCS)C(=O)O]

Further features of the present invention will become apparent from the following description of exemplary embodiments.

### DESCRIPTION OF THE EMBODIMENTS

Embodiments of the present invention are described in detail below, but the technical scope of the present invention is not limited to these embodiments.

### DESCRIPTION ABOUT PROBLEMS

The inventors of the present invention have made investigations by a latex agglutination method, which is one method of detecting a target substance. As a result, it has been recognized that in the solid-phase support described in each of Japanese Patent Application Laid-Open No. 2000-351814 and International Publication WO2007/063616, nonspecific adsorption occurs in some cases particularly when a target substance having a low concentration is detected, that is, in a system in which a target substance in a specimen having a high concentration is detected. In particular, it has been found that further suppression of nonspecific adsorption is required in a linker moiety that bonds a styrene-glycidyl methacrylate polymer and a ligand to each other. A support according to the embodiment of the present invention solves the above-mentioned problems.

### Support

A support according to the present invention includes a substrate, a structure represented by the following formula (1), and a structure having a reactive functional group. The support according to the present invention is a support including: a substrate; and a structure having a reactive functional group bonded to the substrate through a structure represented by the following formula (1), wherein the structure having a reactive functional group has as the reactive functional group at least one selected from the group consisting of: a carboxy group; a maleimide group; a carbamoyl group; a tosyl group; an amino group; an epoxy group; and a thiol group:

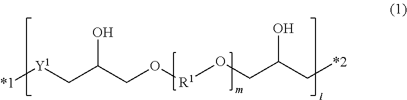

in the formula (1), “1” represents an integer of 1 or more and 11 or less, “m” represents an integer of 1 or more and 10 or less, Y¹ represents a sulfur atom, a structure represented by the following formula (2), or a structure represented by the following formula (3), R¹ represents an unsubstituted alkylene group having 2 to 4 carbon atoms, *1 represents a bonding position to a carbon atom or a sulfur atom in the substrate or the structure having a reactive functional group, *2 represents a bonding position to a nitrogen atom, a sulfur atom, or an oxygen atom in the substrate or the structure having a reactive functional group, provided that when *1 represents a bonding position to an atom in the substrate, *2 represents a bonding position to an atom in the structure having a reactive functional group, and when *1 represents a bonding position to an atom in the structure having a reactive functional group, *2 represents a bonding position to an atom in the substrate;

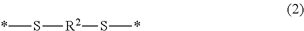

in the formula (2), R² represents an alkylene group having 1 to 8 carbon atoms, and the alkylene group represented by R² may have at least one of a halogen atom and a hydroxy group as a substituent, and * represents a bonding position in the structure represented by the formula (1);

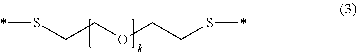

in the formula (3), “k” represents an integer of 1 or more and 8 or less, and * represents a bonding position in the structure represented by the formula (1).

In the structure represented by the formula (1), when “1” represents an integer of 2 or more, the structure may be a different structure for each repeating unit.

The support has the above-mentioned structure as a linker, and hence the substrate and the ligand can be bonded to each other at an appropriate distance while nonspecific adsorption is suppressed.

### Substrate

The substrate in the present invention is a portion that becomes a scaffold in which the ligand serving as a biosensor is immobilized through the linker, and the structure of the substrate is not particularly limited, but it is preferred that the substrate contain a polymer having a structural unit derived from styrenes. In addition, it is preferred that the substrate contain a polymer having a structural unit derived from glycidyl (meth)acrylate. Herein, the expression “glycidyl (meth)acrylate” means “glycidyl methacrylate and/or glycidyl acrylate.” The structural unit derived from glycidyl (meth)acrylate has an action of reducing the nonspecific adsorption to the substrate. The structural unit derived from glycidyl (meth)acrylate is obtained by adding, for example, glycidyl methacrylate or glycidyl acrylate during the production of the support.

In the support of the present invention, the substrate preferably contains a polymer having a structural unit represented by the following formula (4-1):

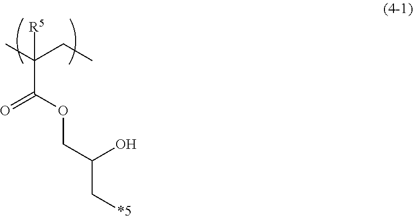

in the formula (4-1), R⁵ represents a hydrogen atom or a methyl group, and *5 represents a bonding position to a sulfur atom of the structure represented by the formula (1).

Meanwhile, the reason why the substrate preferably contains the polymer having a structural unit derived from styrenes is as described below. When the support in the present invention has a particle shape, it is assumed that the support is purified by a method, such as centrifugal separation or ultrafiltration, during modification of a glycidyl group on the surface of the particle or sensitization of a ligand. In this case, the presence of the structural unit derived from styrenes, which has a high glass transition temperature and is excellent in mechanical strength, contributes to the suppression of damage to the particle, such as cracking or chipping. Examples of the styrenes to be added at the time of the synthesis of the support for obtaining the structural unit derived from the styrenes include styrene, α-methylstyrene, β-methylstyrene, o-methylstyrene, m-methylstyrene, p-methylstyrene, 2,4-dimethylstyrene, p-n-butylstyrene, p-tert-butylstyrene, p-n-hexylstyrene, p-n-octylstyrene, p-n-nonylstyrene, p-n-decylstyrene, p-n-dodecylstyrene, p-methoxystyrene, and p-phenylstyrene, but the styrene is not limited thereto to the extent that the object of the present invention can be achieved. In addition, two or more kinds of the styrenes may be used in combination. When the mass of the substrate is set to 100 parts by mass, the content of the styrene is preferably 10 parts by mass or more and 70 parts by mass or less because sufficient strength can be imparted to the particle while the nonspecific adsorption is reduced.

In addition, the substrate of the present invention may further contain a polymer having a repeating unit derived from a radically polymerizable monomer having crosslinkability. Examples of the radically polymerizable monomer having crosslinkability include diethylene glycol diacrylate, triethylene glycol diacrylate, tetraethylene glycol diacrylate, polyethylene glycol diacrylate, 1,6-hexanediol diacrylate, neopentyl glycol diacrylate, tripropylene glycol diacrylate, polypropylene glycol diacrylate, 2,2′-bis(4-(acryloxydiethoxy)phenyl)propane, trimethylolpropane triacrylate, tetramethylolmethane tetraacrylate, ethylene glycol dimethacrylate, diethylene glycol dimethacrylate, triethylene glycol dimethacrylate, tetraethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2,2′-bis(4-(methacryloxydiethoxy)phenyl)propane, 2,2′-bis(4-(methacryloxypolyethoxy)phenyl)propane, trimethylolpropane trimethacrylate, tetramethylolmethane tetramethacrylate, divinylbenzene, divinylnaphthalene, and divinyl ether, but the monomer is not limited thereto to the extent that the object of the present invention can be achieved. In addition, two or more kinds of the radically polymerizable monomers each having crosslinkability may be used in combination.

The shape of the substrate of the present invention is not particularly limited. For example, the support according to the present invention may be applied onto a flat plate as an application example to a biosensor portion of immunochromatography. When the shape of the substrate is a particle, the specific surface area of the surface of the support is maximized, and hence the effect can be most suitably exhibited. A case in which the shape of the substrate of the present invention is a particle indicates that the particle diameter of the particle may be measured by a dynamic light scattering method. The particle diameter thereof is preferably 0.05 μm or more and 1.00 μm or less, more preferably 0.10 μm or more and 0.60 μm or less, still more preferably 0.10 μm or more and 0.40 μm or less in terms of number-average particle diameter. When the particle diameter is 0.10 μm or more and 0.40 μm or less, centrifugal separation for purification can be performed in the dispersion of the particle in an aqueous medium. Accordingly, handling is facilitated, and particle sedimentation hardly occurs during storage in a state of particle dispersion.

The substrate has at least one selected from the group consisting of: a carbon atom; a nitrogen atom; a sulfur atom; and an oxygen atom as an atom that forms a bond to the structure represented by the formula (1).

### <Structure Represented by Formula (1)>

The inventors of the present invention have intensively investigated a partial structure of the linker, and as a result, have arrived at the invention of a highly hydrophilic unit having a sulfide structure, an ether structure, and a hydroxy group.

The structure represented by the formula (1) is defined as the partial structure of the linker between the substrate and the structure having a reactive functional group. When the highly hydrophilic unit having a sulfide structure, an ether structure, and a hydroxy group is incorporated into the linker, the substrate and the ligand can be bonded to each other at an appropriate distance while nonspecific adsorption is suppressed.

In the structure represented by the formula (1), R¹ represents an unsubstituted alkylene group having 2 to 4 carbon atoms. In the structure represented by the formula (1), Y¹ represents a sulfur atom, a structure represented by the formula (2), or a structure represented by the formula (3). When Y¹ represents the structure represented by the formula (2), R² in the structure represented by the formula (2) represents a substituted or unsubstituted alkylene group having 1 to 8 carbon atoms. The alkylene group represented by R² may have at least one of a halogen atom and a hydroxy group as a substituent. The length of the linker may be adjusted by the number of carbon atoms of the alkylene group represented by R¹ or the alkylene group represented by R². However, when the number of carbon atoms of R² exceeds 8 or the number of carbon atoms of R¹ exceeds 4, the hydrophilicity of the linker is impaired, and nonspecific adsorption occurs in some cases. From the viewpoint of ease in synthesis, R¹ preferably represents an alkylene group having 2 carbon atoms. In addition, similarly, when Y¹ represents the structure represented by the formula (2), R² preferably represents an alkylene group having 2 carbon atoms.

In addition, when Y¹ represents the structure represented by the formula (3), “k” in the structure represented by the formula (3) represents an integer of 1 or more and 8 or less, but from the viewpoint of ease in synthesis, “k” preferably represents 1.

In the structure represented by the formula (1), “1” represents an integer of 1 or more and 11 or less, and “m” represents an integer of 1 or more and 10 or less. When “1” and “m” each fall within the above-mentioned ranges, an appropriate distance can be maintained between the substrate and the ligand. In addition, from the viewpoint of ease in synthesis, “1” preferably represents an integer of 1 or more and 6 or less, and “m” preferably represents an integer of 1 or more and 4 or less and “m” more preferably represents 1. The number of carbon atoms of “m” or R¹ in the structure represented by the formula (1) may be adjusted by the kind of a monomer to be added in synthesis of the support or the linker partial structure.

In addition, in the structure represented by the formula (1), *1 represents a bonding position to a carbon atom or a sulfur atom in the substrate, or a bonding position to a carbon atom or a sulfur atom in the structure having a reactive functional group. * 2 represents a bonding position to a nitrogen atom, a sulfur atom, or an oxygen atom in the substrate, or a bonding position to a nitrogen atom, a sulfur atom, or an oxygen atom in the structure having a reactive functional group. When *1 represents a bonding position to an atom in the substrate, *2 represents a bonding position to an atom in the structure having a reactive functional group. When *1 represents a bonding position to an atom in the structure having a reactive functional group, *2 represents a bonding position to an atom in the substrate. That is, it is appropriate that one of *1 and *2 of the structure represented by the formula (1) is bonded to the substrate, and the other is bonded to the structure having a reactive functional group, and a direction is not particularly limited.

<Structure having Reactive Functional Group>

The structure having a reactive functional group has a reactive functional group capable of being chemically bonded to a ligand in the structure, and the reactive functional group is at least one selected from the group consisting of: a carboxy group; a maleimide group; a carbamoyl group; a tosyl group; an amino group; an epoxy group; and a thiol group. The above-mentioned structure having a reactive functional group preferably contains one or two reactive functional groups in the structure so as to have satisfactory reactivity with the ligand.

It is preferred that the support according to the present invention have, as the structure having a reactive functional group, at least one of a structure represented by the following formula (3-1), a structure represented by the following formula (3-2), a structure represented by the following formula (3-3), and a structure represented by the following formula (3-4):

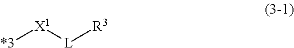

in the formula (3-1), L represents a substituted or unsubstituted alkylene group having 1 to 6 carbon atoms, the group having bonding sites to be bonded to X¹ and R³, and the alkylene group represented by L may have at least one of a halogen atom and a hydroxy group as a substituent, X¹ represents an imino group, a sulfur atom, or an oxygen atom, *3 represents a bonding position to a carbon atom of the structure represented by the formula (1), and R³ represents the reactive functional group;

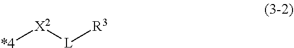

in the formula (3-2), L represents a substituted or unsubstituted alkylene group having 1 to 6 carbon atoms, the group having bonding sites to be bonded to X² and R³, and the alkylene group represented by L may have at least one of a halogen atom and a hydroxy group as a substituent, X² represents a methylene group or a sulfur atom, *4 represents a bonding position to a sulfur atom of the structure represented by the formula (1), and R³ represents the reactive functional group;

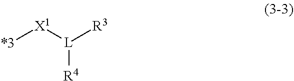

in the formula (3-3), L represents a substituted or unsubstituted trivalent saturated hydrocarbon group having 1 to 6 carbon atoms, the group having bonding sites to be bonded to X¹, R³, and R⁴, and the saturated hydrocarbon group represented by L may have at least one of a halogen atom and a hydroxy group as a substituent, X¹ represents an imino group, a sulfur atom, or an oxygen atom, *3 represents a bonding position to a carbon atom of the structure represented by the formula (1), and R³ and R⁴ each represent the reactive functional group;

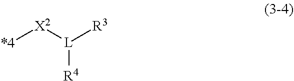

in the formula (3-4), L represents a substituted or unsubstituted trivalent saturated hydrocarbon group having 1 to 6 carbon atoms, the group having bonding sites to be bonded to X², R³, and R⁴, and the saturated hydrocarbon group represented by L may have at least one of a halogen atom and a hydroxy group as a substituent, X² represents a methylene group or a sulfur atom, *4 represents a bonding position to a sulfur atom of the structure represented by the formula (1), and R³ and R⁴ each represent the reactive functional group.

In the formula (3-1), the formula (3-2), the formula (3-3), and the formula (3-4), the reactive functional group represented by each of R³ and R⁴ may be bonded to any carbon atom of the alkylene group or the saturated hydrocarbon group represented by L. The structure having a reactive functional group has at least one selected from the group consisting of: a carbon atom; a nitrogen atom; a sulfur atom; and an oxygen atom as an atom that forms a bond to the structure represented by the formula (1). It is not required that all reactive functional groups in the support be bonded to the structure represented by the formula (1), and the support may have a reactive functional group not bonded to the structure represented by the formula (1).

<Compounds for Use in Formation of Structure represented by Formula (1) and Structure having Reactive Functional Group>

For example, a compound represented by the following formula (5-1) and/or a compound represented by the following formula (5-2) may be used so that the support contains the structure represented by the formula (1) and the structure having a reactive functional group.

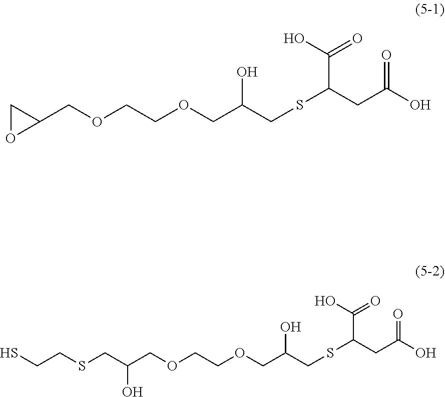 [SMILES: O=C(O)CC(SCC(O)COCCOCC1CO1)C(=O)O ; O=C(O)CC(SCC(O)COCCOCC(O)CSCCS)C(=O)O]

A method of producing the support may include a step of synthesizing at least one of the above-mentioned compounds as the linker partial structure for use in the formation of the structure represented by the formula (1) and the structure having a reactive functional group. After the above-mentioned step, the support may be obtained through a step of causing the above-mentioned compound to react with, for example, a compound for use in the formation of the substrate.

### <Measurement of Molecular Weight by GPC>

For the structure represented by the formula (1), a molecular weight may be measured by GPC. An example of a method for the measurement is as described below.

Methanol for high-performance liquid chromatography and a sample are placed in a vial and the sample is dissolved. For example, a sample containing a compound for use in the formation of the structure represented by the formula (1) (e.g., a compound corresponding to the structure represented by the formula (1)) is used as the sample. After the dissolution of the sample is recognized, filtration is performed through use of a disposable disk filter manufactured by Tosoh Corporation (product name: Myshori Disk, aperture: 0.5 μm), and a substance passed through the filter is defined as a GPC sample.

A sample solution is adjusted so that its concentration is about 1.0 mass %.

Measurement is performed through use of this sample solution under the following conditions.
Apparatus: Waters APC System (Nihon Waters K. K.)
Detector: RI, PDA
Column: ACQUITY APC XT900 (150 mm), XT200 (75 mm), XT125 (75 mm), XT45 (150 mm)
Temperature: 40.0° C.
Solvent: methanol for high-performance liquid chromatography
Flow rate: 0.8 mL/min
Injection volume: 10 μL

At the time of the measurement of a peak molecular weight, a molecular weight calibration curve produced by using the following standard polyethylene glycol resin may be used.
Standard polyethylene glycol resin: product name: “POLYETHYLENE GLYCOL Easi Vial” (Agilent Technologies, Inc.)

### <Ligand>

The ligand in the present invention refers to be a compound that is specifically bonded to a receptor that a specific target substance has. The site at which the ligand is bonded to the target substance is predetermined, and the ligand has a selectively or specifically high affinity for the target substance. Examples thereof include: an antigen and an antibody; an enzyme protein and a substrate or a receptor thereof; a signal substance, such as a hormone or a neurotransmitter, and a receptor thereof; and a nucleic acid. However, the ligand in the present invention is not limited thereto.

### <Ligand-bonded Support>

A ligand-bonded support in the present invention is a support in which the ligand and the reactive functional group in the support are bonded to each other, and means a support in a state in which the substrate and the ligand are bonded to each other through the linker at an appropriate distance. That is, the ligand-bonded support in the present invention is the above-mentioned support to which the ligand is chemically bonded through the reactive functional group. A mode of bonding between the reactive functional group and the ligand is not particularly limited, but the reactive functional group is preferably selected from a carboxy group, an amino group, a thiol group, and a maleimide group in terms of selective bonding property to the ligand. Moreover, a carboxy group is most preferably selected in terms of ease in reaction. As a method for a chemical reaction by which a carboxy group and the ligand are chemically bonded to each other, a hitherto known method may be applied to the extent that the object of the present invention can be achieved. For example, a carbodiimide-mediated reaction or an NHS ester activation reaction is a frequently used chemical reaction method, but the method of the present invention for the chemical reaction by which the carboxy group and the ligand are chemically bonded to each other is not limited thereto.

### <Affinity Particle>

In an affinity particle in the present invention, the ligand is chemically bonded to the support in which the shape of the substrate is a particle through the reactive functional group. In the ligand-bonded support in the present invention, when the shape of the substrate is a particle, the specific surface area of the surface of the support is maximized, and hence the effect can be most suitably exhibited. The ligand-bonded support in which the shape of the substance is a particle is a particle having a selectively or specifically high affinity for a target substance, and hence the ligand-bonded support may be defined as an affinity particle. The ligand-bonded support in which the shape of the substance in the present invention is a particle, that is, the affinity particle can be extremely preferably applied to a method of detecting a target substance in a specimen in in vitro diagnosis, and an example thereof may be application as a particle for a latex agglutination method. When a general particle is used as a particle for a latex agglutination method, an antigen (antibody) that is the target substance, foreign matter in serum or plasma, or the like nonspecifically adsorbs to the surface of the particle, and hence concern is raised in that unintended interparticle agglutination occurs owing to the adsorption to impair the accuracy of an immunological test. When the affinity particle according to the present invention is used, unintended interparticle agglutination due to nonspecific adsorption can be reduced, and the target substance having a low concentration can be detected with high sensitivity.

### <Reagent for Use in Detection of Target Substance in Specimen by In Vitro Diagnosis>

A reagent for use in detection of a target substance in a specimen by in vitro diagnosis contains the ligand-bonded support (affinity particle) according to the present invention. The reagent according to the present invention may include two or more constituent reagents. For example, when a first reagent serving as a specimen diluent and a second reagent serving as a latex test solution are set, the affinity particle according to the present invention may be incorporated into the second reagent. The amount of the affinity particle to be incorporated into the reagent is preferably from 0.001 mass % to 20 mass %, more preferably from 0.01 mass % to 10 mass %.

In addition, the reagent according to the present invention may contain a third substance, such as a buffer or a water-soluble additive, in addition to the affinity particle according to the present invention to the extent that the object of the present invention can be achieved. Examples of the buffer to be used for the reagent according to the present invention include, but not limited to, Tris hydrochloric acid, boric acid, phosphoric acid, acetic acid, citric acid, succinic acid, phthalic acid, glutaric acid, maleic acid, glycine, and salts thereof, and Good's buffers, such as MES, Bis-Tris, ADA, PIPES, ACES, MOPSO, BES, MOPS, TES, and HEPES. In addition, a water-soluble additive, for example, a water-soluble polymer, such as polyethylene glycol, carboxymethyl cellulose, methyl cellulose, dextran, polyvinylpyrrolidone, polyglycosylethyl methacrylate, pullulan, dextran, or elsinan, may be incorporated thereinto in order to increase the sensitivity of the reaction. Further, for example, a protein, such as casein or gelatin, or a decomposition product thereof or a modified product thereof, a quaternary ammonium salt such as choline chloride, EDTA, a polyanion, a chaotropic ion (e.g., Cl⁻, I⁻, or SCN⁻), an amino acid, a surfactant, and a sugar may be incorporated thereinto for the purposes of, for example, an improvement in specificity and an improvement in stability of the reagent.

### <Kit for Use in Detection of Target Substance in Specimen by In Vitro Diagnosis>

A kit for use in detection of a target substance in a specimen by in vitro diagnosis includes a cartridge including the ligand-bonded support (affinity particle) according to the present invention.

The cartridge indicates a container having a detection function by itself or a container to be installed in an apparatus. An example of the container having a detection function by itself is a device for immunochromatography in which the affinity particle is supported. An example of the container to be installed in an apparatus is a container capable of being installed in an automatic analyzer.

In addition, the kit of the present invention may include a positive control, a negative control, and the like in addition to the above-mentioned cartridge. In addition to serum or physiological saline free of any target substance that may be measured, a buffer-containing aqueous solution may be used as a medium for the positive control or the negative control. The kit according to the present invention may be used in the method of detecting a target substance according to the present invention similarly to a typical kit for use in detection of a target substance in a specimen by in vitro diagnosis. In addition, the concentration of the target substance may be measured by a hitherto known method. In particular, the method is suitable for the detection of the target substance in the specimen by a latex agglutination method. In addition, the kit according to the present invention may include an instruction for use, a tool for collecting a specimen (e.g., a pipette for collection, a syringe, a cotton swab, or a filtration filter), and a specimen extract liquid in addition to the foregoing.

### <Detection Method>

The method of detecting a target substance in a specimen by in vitro diagnosis according to the present invention is characterized by including a step of mixing a reagent containing the ligand-bonded support (affinity particle) according to the present invention and the specimen that may contain the target substance. The reagent containing the affinity particle according to the present invention and the specimen are preferably mixed at a pH in the range of from 3.0 to 11.0. In addition, a mixing temperature falls within the range of from 20° C. to 50° C., and a mixing time falls within the range of from 1 minute to 20 minutes. In addition, in this detection method, a solvent is preferably used. In addition, the concentration of the affinity particle in this detection method is preferably from 0.001 mass % to 5 mass %, more preferably from 0.01 mass % to 1 mass % in a reaction system. In the detection method according to the present invention, when the interparticle agglutination caused as a result of mixing the affinity particle according to the present invention and a specimen is optically detected, the target substance in the specimen is detected, and the concentration of the target substance can also be measured. In a method of optically detecting an agglutination reaction, an optical instrument that can detect a scattered light intensity, a transmitted light intensity, an absorbance, and the like only needs to be used to measure the variations of these values. Specifically, the detection method according to the present invention may include a step of measuring the agglutination of the affinity particle by a change in absorbance to detect the presence or absence or the concentration of the target substance.

### EXAMPLES

The present invention is described in detail below by way of Examples.

However, the present invention is not limited to these Examples.

### Partial Structure Synthesis Example 1

### Synthesis of Linker Partial Structure 1

3.0 g (17.2 mmol) of ethylene glycol diglycidyl ether (FUJIFILM Wako Pure Chemical Corporation) and 5.0 g of ion-exchanged water were weighed in a 100 mL recovery flask equipped with a dropping funnel. 2.8 g (18.1 mmol) of mercaptosuccinic acid (FUJIFILM Wako Pure Chemical Corporation) and 16.0 g of ion-exchanged water were weighed in a 30 mL vial, and 4.2 g of triethylamine (Kishida Chemical Co., Ltd.) was added thereto to prepare 31.0 g of a mixed liquid having a pH of 10. Next, while the contents of the recovery flask were placed in an ice bath and then stirred at 500 rpm, the above-mentioned mixed liquid was added dropwise through use of the dropping funnel over 2 hours. After the completion of the dropwise addition, a reaction was performed for 4 hours in the ice bath, and then a reaction was further performed at room temperature for 16 hours to provide a dissolved liquid of a linker partial structure 1.

Part of the resultant dissolved liquid of the linker partial structure 1 was freeze-dried, purified by HPLC, and subjected to ¹H-NMR measurement and liquid chromatography (LC-MS). Thus, the presence of a structure corresponding the structure represented by the formula (1) in which 1=1 (a compound represented by the formula (5-1)) was recognized.

### Partial Structure Synthesis Example 2

### Synthesis of Linker Partial Structure 2

1.5 g (15.9 mmol) of 1,2-ethanedithiol (Tokyo Chemical Industry Co., Ltd.) and 6.0 g of ion-exchanged water were weighed in a 100 mL recovery flask equipped with a dropping funnel. Next, 31.0 g of the dissolved liquid of the linker partial structure 1 (containing 16.3 mmol of the linker partial structure 1 as a theoretical value) obtained in the foregoing was added dropwise through use of the dropping funnel over 2 hours while the contents were stirred at room temperature and 500 rpm. After the completion of the dropwise addition, a reaction was further performed for 7 hours to provide a dissolved liquid of a linker partial structure 2.

Part of the resultant dissolved liquid of the linker partial structure 2 was centrifuged, and the supernatant was recovered, freeze-dried, purified by HPLC, and subjected to ¹H-NMR measurement and liquid chromatography (LC-MS). Thus, the presence of a structure corresponding the structure represented by the formula (1) in which 1=1 (a compound represented by the formula (5-2)) was recognized.

### Partial Structure Synthesis Example 3

### Synthesis of Linker Partial Structure 3

The following materials were weighed in a 50 mL recovery flask equipped with a dropping funnel.
2.0 g (11.5 mmol) of ethylene glycol diglycidyl ether (FUJIFILM Wako Pure Chemical Corporation).
1.3 g (13.7 mmol) of 1,2-ethanedithiol (Tokyo Chemical Industry Co., Ltd.)
4.0 mL of methanol (Kishida Chemical Co., Ltd.)

Next, while the contents of the recovery flask were stirred at room temperature and 500 rpm, 4.8 mL of triethylamine (Kishida Chemical Co., Ltd.) was added dropwise through use of the dropping funnel. After the completion of the dropwise addition, a reaction was further performed for 18 hours to produce a dissolved liquid of an intermediate of a linker partial structure 3.

The resultant dissolved liquid of the intermediate of the linker partial structure 3 was freeze-dried to provide the intermediate of the linker partial structure 3.

The peak molecular weight of the resultant intermediate of the linker partial structure 3 was 1.4×10³. When the molecular weight of ethylene glycol diglycidyl ether was set to 174.2 and the molecular weight of 1,2-ethanedithiol was set to 94.2, the presence of the structure represented by the formula (1) in which 1=5 was recognized. In the measurement of the peak molecular weights of intermediates of the linker partial structure 3 and a linker partial structure 4 described later, the measurement was performed by the method described in the section <Measurement of Molecular Weight by GPC> described above.

Next, the whole amount of the above-mentioned intermediate of the linker partial structure 3 and 4.0 mL of methanol (Kishida Chemical Co., Ltd.) were weighed in a 50 mL recovery flask equipped with a dropping funnel. 4.7 g of the dissolved liquid of the linker partial structure 1 (containing 2.47 mmol of the linker partial structure 1) obtained in the foregoing was added dropwise through use of the dropping funnel over 1 hour. After the completion of the dropwise addition, a reaction was further performed for 18 hours to produce a dissolved liquid of the linker partial structure 3.

### Partial Structure Synthesis Example 4

### Synthesis of Linker Partial Structure 4

An intermediate of a linker partial structure 4 was synthesized in the same manner as that for the intermediate of the linker partial structure 3 except that: the amount of ethylene glycol diglycidyl ether (FUJIFILM Wako Pure Chemical Corporation) was changed to 4.0 g (23.0 mmol); the amount of 1,2-ethanedithiol (Tokyo Chemical Industry Co., Ltd.) was changed to 2.4 g (25.3 mmol); the amount of methanol (Kishida Chemical Co., Ltd.) was changed to 8.0 mL; and the amount of triethylamine (Kishida Chemical Co., Ltd.) was changed to 8.8 mL.

The peak molecular weight of the resultant intermediate of the linker partial structure 4 was 2.8×10³. When the molecular weight of ethylene glycol diglycidyl ether was set to 174.2 and the molecular weight of 1,2-ethanedithiol was set to 94.2, the presence of the structure represented by the formula (1) in which 1=10 was recognized.

After that, the linker partial structure 4 was synthesized in the same manner as in Partial Structure Synthesis Example 3 except that the intermediate of the linker partial structure 4 was used instead of the intermediate of the linker partial structure 3.

### Particle Synthesis Example 1

### Synthesis of Particulate Copolymer 1

The following materials were weighed in a 2 L four-necked separable flask to provide a mixed liquid.
22.7 g of styrene (St: Kishida Chemical Co., Ltd.)
33.9 g of glycidyl methacrylate (GMA: Kishida Chemical Co., Ltd.)·
0.86 g of divinylbenzene (DVB: Kishida Chemical Co., Ltd.)
2,168.6 g of ion-exchanged water

After that, the mixed liquid was held at 70° C. while being stirred at 200 rpm, and nitrogen was flowed at a flow rate of 200 mL/min to remove oxygen from the inside of the four-necked separable flask. Next, a separately prepared dissolved liquid, which had been obtained by dissolving 1.13 g of V-50 (FUJIFILM Wako Pure Chemical Corporation) in 30 g of ion-exchanged water, was added to the mixed liquid to initiate soap-free emulsion polymerization. Two hours after the initiation of the polymerization, 5.8 g of GMA was added to the four-necked separable flask, and the mixture was further held at 70° C. while being stirred for 22 hours at 200 rpm. Thus, an aqueous dispersion containing a particulate copolymer 1 was obtained. After the dispersion had been gradually cooled to room temperature, part of the dispersion was collected, and its polymerization conversion ratio was evaluated by using proton NMR, gas chromatography, and gel permeation chromatography. As a result, it was recognized that the polymerization conversion ratio was substantially 100%. The number-average particle diameter of the particulate copolymer 1 was 206.9 nm. The particulate copolymer 1 was subjected to ultrafiltration concentration, or was diluted by the addition of ion-exchanged water, so as to provide a 2.5 mass % aqueous dispersion, and the dispersion was stored under a light-shielding condition at 4° C. The above-mentioned polymerization conversion ratio was calculated from the amount of monomers loaded in the polymerization process. The solution after the polymerization reaction was analyzed by using gas chromatography or the like, the amount of the remaining monomers was determined, and a value indicating the ratio of the monomers converted to a polymer by the polymerization reaction (polymerization conversion ratio) was determined from the amount of the remaining monomers. In this case, when the amount of the remaining monomers was a detection limit or less, the polymerization conversion ratio was set to substantially 100% (all the monomers were polymerized).

A method of evaluating a number-average particle diameter was performed by the following method based on a dynamic light scattering method. Particles were dispersed in ion-exchanged water having an electric conductivity of 10 μS/cm or less so that their concentration was 0.001 mass %, and the measurement was performed with ZETASIZER (Nano-ZS: Spectris Co., Ltd.) at 25° C. With regard to analysis parameters, the refractive index of latex (n=about 1.59) was selected as the refractive index of the particles, and pure water was selected as a solvent. The measurement was performed ten times, and the average of the ten measured values was adopted as the particle diameter in water.

### Synthesis of Particle Precursor 1

The following materials were weighed in a 100 mL round-bottom flask, and triethylamine (Kishida Chemical Co., Ltd.) was added to the mixture to adjust its pH to 10.
24 g of the 2.5 mass % aqueous dispersion of the particulate copolymer 1
3.3 g of ion-exchanged water
0.044 mL (0.41 mmol) of dimercaptoethanol (FUJIFILM Wako Pure Chemical Corporation).
0.187 mL (1.65 mmol) of 3-mercapto-1,2-propanediol (FUJIFILM Wako Pure Chemical Corporation)

Next, the temperature of the contents of the round-bottom flask was increased to 70° C. while the contents were stirred at 200 rpm. Further, the contents were held in this state for 18 hours to provide a dispersion of a particle precursor 1. The particle precursor 1 was separated from the dispersion with a centrifugal separator, and the particle precursor 1 was re-dispersed in ion-exchanged water; the operation was repeated five times to purify the particle precursor 1, which was stored in the state of an aqueous dispersion in which the concentration of the particle precursor 1 was finally adjusted to 2.5 mass %. Storage conditions were set to 4° C. under a light-shielding condition.

### Synthesis of Particles 1

24 g of the 2.5 mass % aqueous dispersion of the particle precursor 1, 3.3 g of ion-exchanged water, and 132.9 mg (0.41 mmol) of the linker partial structure dissolved liquid containing the linker partial structure 1 were weighed in a 100 mL round-bottom flask. Triethylamine (Kishida Chemical Co., Ltd.) was added to the mixture to adjust its pH to 10. Next, the temperature of the contents of the round-bottom flask was increased to 70° C. while the contents were stirred at 200 rpm. Further, the contents were held in this state for 18 hours to provide a dispersion of particles 1. The particles were separated from the dispersion with a centrifugal separator, and the particles were re-dispersed in ion-exchanged water; the operation was repeated five times to purify the particles, which were stored in the state of an aqueous dispersion in which the concentration of the particles 1 was finally adjusted to 5.0 mass %. Storage conditions were set to 4° C. under a light-shielding condition. Reactive functional groups in the particles 1 are a thiol group (a residue derived from dimercaptoethanol), an epoxy group (a residue derived from glycidyl methacrylate), and a carboxy group (derived from mercaptosuccinic acid).

### Particle Synthesis Example 2

### Synthesis of Particles 2

The following materials were weighed in a 100 mL round-bottom flask, and triethylamine (Kishida Chemical Co., Ltd.) was added to the mixture to adjust its pH to 10.
24 g of the 2.5 mass % aqueous dispersion of the particulate copolymer 1
3.3 g of ion-exchanged water
171.6 mg (0.41 mmol) of the linker partial structure dissolved liquid containing the linker partial structure 2
0.214 mL (2.34 mmol) of 3-mercapto-1,2-propanediol (FUJIFILM Wako Pure Chemical Corporation)

Next, the temperature of the contents of the round-bottom flask was increased to 70° C. while the contents were stirred at 200 rpm. Further, the contents were held in this state for 18 hours to provide a dispersion of particles 2. The particles 2 were separated from the dispersion with a centrifugal separator, and the particles 2 were re-dispersed in ion-exchanged water; the operation was repeated five times to purify the particles 2, which were stored in the state of an aqueous dispersion in which the concentration of the particles 2 was finally adjusted to 5.0 mass %. Storage conditions were set to 4° C. under a light-shielding condition. Reactive functional groups in the particles 2 are an epoxy group (a residue derived from glycidyl methacrylate) and a carboxy group (derived from mercaptosuccinic acid).

### Particle Synthesis Example 3

### Synthesis of Particle Precursor 3

A dispersion of a particle precursor 3 was obtained in the same manner as that for the particle precursor 1 except: that the amount of dimercaptoethanol (FUJIFILM Wako Pure Chemical Corporation) was changed to 0.11 mL (1.03 mmol); and the amount of 3-mercapto-1,2-propanediol (FUJIFILM Wako Pure Chemical Corporation) was changed to 0.117 mL (1.03 mmol).

### Synthesis of Particles 3

A dispersion of particles 3 was obtained in the same manner as that for the particles 1 except that: the particle precursor 3 was used instead of the particle precursor 1; and the amount of the linker partial structure 1 was changed to 332.3 mg (1.03 mmol). Reactive functional groups in the particles 3 are a thiol group (a residue derived from dimercaptoethanol), an epoxy group (a residue derived from glycidyl methacrylate), and a carboxy group (derived from mercaptosuccinic acid).

### Particle Synthesis Example 4

### Synthesis of Particles 4

A dispersion of particles 4 was obtained in the same manner as that for the particles 2 except that 721.8 mg (0.41 mmol) of a linker partial structure dissolved liquid containing the linker partial structure 3 was used instead of the linker partial structure dissolved liquid containing the linker partial structure 2. Reactive functional groups in the particles 4 are an epoxy group (a residue derived from glycidyl methacrylate) and a carboxy group (derived from mercaptosuccinic acid).

### Particle Synthesis Example 5

### Synthesis of Particles 5

A dispersion of particles 5 was obtained in the same manner as that for the particles 2 except that 1,271.9 mg (0.41 mmol) of a linker partial structure dissolved liquid containing the linker partial structure 4 was used instead of the linker partial structure dissolved liquid containing the linker partial structure 2. Reactive functional groups in the particles 5 are an epoxy group (a residue derived from glycidyl methacrylate) and a carboxy group (derived from mercaptosuccinic acid).

### Particle Synthesis Example 6

### Synthesis of Particles 6

The following materials were weighed in a 100 mL round-bottom flask, and triethylamine (Kishida Chemical Co., Ltd.) was added to the mixture to adjust its pH to 11.
6.0 g of a 10 mass % aqueous dispersion of polystyrene particles (manufactured by Magsphere Inc., carboxy group modification type, CA200NM)·
3.3 g of ion-exchanged water
132.9 mg (0.41 mmol) of the linker partial structure dissolved liquid containing the linker partial structure 1

Next, the temperature of the contents of the round-bottom flask was increased to 85° C. while the contents were stirred at 200 rpm. Further, the contents were held in this state for 18 hours to provide a dispersion of particles 6. The particles were separated from the dispersion with a centrifugal separator, and the particles were re-dispersed in ion-exchanged water; the operation was repeated five times to purify the particles, which were stored in the state of an aqueous dispersion in which the concentration of the particles 6 was finally adjusted to 5.0 mass %. Storage conditions were set to 4° C. under a light-shielding condition. Reactive functional groups in the particles 6 are carboxy groups (derived from polystyrene particles and derived from mercaptosuccinic acid).

In the particles 1 to 6, “m” represents 1 and R¹ represents an alkylene group having 2 carbon atoms in the structure represented by the formula (1). [Comparative Particle Synthesis Example 1]

### Synthesis of Particle Precursor 7-1

The 2.5 mass % aqueous dispersion of the particulate copolymer 1 obtained in Particle Synthesis Example 1 was concentrated to a 10 mass % aqueous dispersion with a centrifugal separator to give a concentrated dispersion. 20 g of the concentrated dispersion was weighed in a 200 mL round-bottom flask. Under a state in which the concentrated dispersion was held at 4° C., 28% ammonia water containing 50-fold mol of ammonia (Kishida Chemical Co., Ltd.) with respect to the theoretical amount of the GMA-derived epoxy groups in the 20 g of the concentrated dispersion was added thereto. The pH was adjusted to 11 by using a 2 N hydrochloric acid aqueous solution and a 2 N sodium hydroxide aqueous solution. After that, the temperature of the contents of the round-bottom flask was increased to 70° C. while the contents were stirred at 100 rpm. The contents were held in this state for 24 hours to provide a dispersion of a particle precursor 7-1. The particle precursor 7-1 was separated from the dispersion with a centrifugal separator, and the particle precursor 7-1 was re-dispersed in ion-exchanged water; the operation was repeated eight times, and the resultant was stored in the state of an aqueous dispersion in which the concentration of the particles was finally adjusted to 10 mass %. Storage conditions were set to 4° C. under a light-shielding condition.

### Synthesis of Particle Precursor 7-2

6.3 g of the 10 wt % aqueous dispersion of the particle precursor 7-1 and 3.81 g of ethylene glycol diglycidyl ether (Tokyo Chemical Industry Co., Ltd.) were weighed in a 50 mL round-bottom flask, and the pH of the mixture was adjusted to 9 by using a 0.1 N hydrochloric acid aqueous solution and a 0.1 N sodium hydroxide aqueous solution. After that, the temperature of the contents of the round-bottom flask was increased to 30° C. while the contents were stirred at 100 rpm. The contents were held in this state for 24 hours to provide a dispersion of a particle precursor 7-2. The particles were separated from the dispersion with a centrifugal separator, and the particles were re-dispersed in ion-exchanged water; the operation was repeated eight times to purify the particles, which were stored in the state of an aqueous dispersion in which the concentration of the particle precursor 7-2 was finally adjusted to 10 mass %. Storage conditions were set to 4° C. under a light-shielding condition.

### Synthesis of Particle Precursor 7-3

The following materials were placed in a 50 mL round-bottom flask, and the pH was adjusted to 11 by using a 1 N hydrochloric acid aqueous solution and a 1 N sodium hydroxide aqueous solution.
6.0 g of the 10 mass % aqueous dispersion of the particle precursor 7-2
A 28% ammonia aqueous solution containing 10-fold mol of ammonia with respect to the theoretical amount of ethylene glycol diglycidyl ether-derived epoxy groups of the particles in the aqueous dispersion (assuming that 100% of the glycidyl methacrylate-derived epoxy groups of the particulate copolymer 1 had reacted with ammonia to be transformed into primary amines, and further assuming that 100% of the primary amines had reacted with one of the two terminal epoxy groups of ethylene glycol diglycidyl ether)

After that, the temperature of the contents of the round-bottom flask was increased to 70° C. while the contents were stirred at 100 rpm. The contents were held in this state for 24 hours to provide a dispersion of a particle precursor 7-3. The particles were separated from the dispersion with a centrifugal separator, and the particles were re-dispersed in ion-exchanged water; the operation was repeated eight times to purify the particles, which were stored in the state of an aqueous dispersion in which the concentration of the particle precursor 7-3 was finally adjusted to 10 mass %. Storage conditions were set to 4° C. under a light-shielding condition.

### Synthesis of Particles 7

Particles were separated from the 10 wt % aqueous dispersion of the particle precursor 7-3 with a centrifugal separator, and the particles were re-dispersed in methanol; the operation was repeated three times to prepare a methanol dispersion of the particles in which the concentration of the particle precursor 7-3 was finally adjusted to 1 mass %. 63 g of the methanol dispersion and 2.77 g of succinic anhydride (Tokyo Chemical Industry Co., Ltd.) were weighed in a 200 mL round-bottom flask, the temperature of the contents of the round-bottom flask was increased to 30° C. while the contents were stirred at 100 rpm. The contents were held in this state for 5 hours to provide a dispersion of particles 7. The particles were separated from the dispersion with a centrifugal separator, and the particles were re-dispersed in ion-exchanged water; the operation was repeated eight times to purify the particles, which were stored in the state of an aqueous dispersion in which the concentration of the particles 7 was finally adjusted to 5.0 mass %.

### Comparative Particle Synthesis Example 2

### Synthesis of Particles 8

The 2.5 mass % aqueous dispersion of the particulate copolymer 1 obtained in Particle Synthesis Example 1 was concentrated to a 10 mass % aqueous dispersion with a centrifugal separator to give a concentrated dispersion. 20 g of the concentrated dispersion was weighed in a 200 mL round-bottom flask. Under a state in which the concentrated dispersion was held at 4° C., 10-fold mol of glycine (Kishida Chemical Co., Ltd.) with respect to the theoretical amount of the GMA-derived epoxy groups in the 20 g of the concentrated dispersion was added thereto, and the pH of the mixture was adjusted to 11 by using a 0.1 N hydrochloric acid aqueous solution and a 0.1 N sodium hydroxide aqueous solution. After that, the temperature of the contents of the round-bottom flask was increased to 70° C. while the contents were stirred at 100 rpm. The contents were held in this state for 24 hours to provide a dispersion of particles 8. The particles were separated from the dispersion with a centrifugal separator, and the particles were re-dispersed in ion-exchanged water; the operation was repeated eight times to purify the particles, which were stored in the state of an aqueous dispersion in which the concentration of the particles 8 was finally adjusted to 5.0 mass %. Storage conditions were set to 4° C. under a light-shielding condition.

### EXAMPLES

(Evaluation 1: Evaluation of Nonspecific Adsorption with respect to Particles)

The following evaluation was performed as the evaluation of nonspecific adsorption with respect to particles for the resultant particles 1 to particles 8.

The particles 1 to the particles 8 were each dispersed in a phosphate buffer (containing 0.01% Tween 20) at 0.1 mass % to prepare a dispersion (liquid P). Next, 55 μL of a diluted specimen liquid (liquid Q) formed of a human normal specimen (serum specimen, 5 μL) and a phosphate buffer (50 μL) was added to 50 μL of each dispersion, and the absorbance at a wavelength of 572 nm of the mixed liquid immediately after its stirring was measured. A spectrophotometer GeneQuant 1300 manufactured by Biochrom was used in the absorbance measurement. Then, each mixed liquid was left at rest at 37° C. for 5 minutes, and then its absorbance at a wavelength of 572 nm was measured again, followed by the calculation of the value of “(variation AABS in absorbance)×10,000.” When particles having a large value of “(variation AABS in absorbance)×10,000” in this evaluation are used as particles for a latex agglutination method in a specimen test, concern is raised in that a normal specimen is interpreted as being false positive owing to noise. The results of evaluation at the following viewpoint are summarized in Table 1.
AA: ΔABS×10,000 was less than 30, and no change was observed.
A: ΔABS×10,000 was 30 or more and less than 100, and no change was observed.
B: ΔABS×10,000 was 100 or more and less than 1,000, and agglutination was observed.
C: ΔABS×10,000 was 1,000 or more, and intensive agglutination was observed.

### (Production of Affinity Particles by Antibody Sensitization of Particles)

180 μL of a 1.7 mass % water suspension of each of the particles 1 to 5, 7, and 8 was dispensed in a 1.5 mL microtube. 90 μL of a 5.0% aqueous solution of 1-(3-dimethylaminopropyl)-3-ethylcarbodiimide hydrochloride and 90 μL of a 5.0% aqueous solution of N-hydroxysulfosuccinimide sodium salt were added thereto. The mixture was stirred at room temperature for 30 minutes. Thus, a dispersion of particles each having an activated carboxy group (activated particle dispersion) was obtained.

After centrifugal washing, 270 μL of phosphate buffered saline (hereinafter referred to as “PBS”) having a pH of 7.2 was added to the resultant, and the particles each having an activated carboxy group were dispersed with an ultrasonic wave.

5 μL of a 15.0 mg/mL dispersion of clone C5 (Funakoshi Co., Ltd.) of a monoclonal mouse anti-human C-reactive protein (hereinafter referred to as “CRP antibody”) was added thereto. The mixture was stirred at room temperature for 3 hours to provide affinity particles by antibody sensitization of the particles.

Those affinity particles were subjected to centrifugal washing. After that, 1 mL of the PBS was added to the resultant, and the affinity particles were stored in a dispersed state.

0.1 mL (1 mg in terms of particles) of the particle dispersion (solution having a concentration of 1.0 mass %, 10 mg/mL) of the particles 6 was transferred to a microtube (volume: 1.5 mL). 0.12 mL of a buffer (10 mM HEPES, pH: 7.9) was added thereto, and the mixture was centrifuged at 4° C. and 15,000 rpm (20,400 g) for 5 minutes. After the centrifugation, the supernatant was discarded. 0.12 mL of a buffer (25 mM MES, pH: 6.0) was added to the residue, and the particles were re-dispersed with an ultrasonic wave. The centrifugation and the re-dispersion were repeated once, the resultant was further centrifuged at 4° C. and 15,000 rpm (20,400 g) for 5 minutes, and the supernatant was discarded. 50 μL of a buffer (10 mM HEPES, pH: 7.9) was added thereto, 50 μL of a KL polyclonal antibody (Cosmo Bio Co., Ltd.) solution (solution obtained by diluting an antibody with an immobilization buffer so that its concentration became 25 μg/50 μL) was then added thereto, and the mixture was left at 4° C. overnight. Next, the mixture was subjected to centrifugal washing three times with a buffer containing 0.1% BSA to perform blocking treatment. After that, the resultant was centrifuged at 4° C. and 15,000 rpm (20,400 g) for 5 minutes, and the supernatant was discarded. 0.2 mL of a washing buffer (10 mM HEPES, pH: 7.9) was added to the residue, and the particles were dispersed with an ultrasonic wave. A washing operation (the centrifugation and the re-dispersion) with the washing buffer (10 mM HEPES, pH: 7.9) was repeated once. A washing operation was performed with 0.2 mL of a storage buffer (10 mM HEPES, pH: 7.9, containing 0.01% Tween 20) once. 1.0 mL of the storage buffer was added to the washed product, and the particles were dispersed with an ultrasonic wave. The particle concentration of the dispersion finally became 0.1 mass % (1 mg/mL). The dispersion was stored in a refrigerator.

The names of affinity particles are hereinafter represented as “affinity particles 1” and the like directly after the particle names, but in Table 1, affinity particles are also referred to as “particles” as abbreviated names.

### (Evaluation 2: Evaluation of Antibody Sensitization Ratio of Affinity Particles)

The antibody sensitization ratio (%) of the produced affinity particles was determined by protein determination. Herein, the term “antibody sensitization ratio (%)” means the ratio of the amount of the antibody bonded to the particles to the amount of the antibody used in the reaction with the particles (antibody loading amount). An evaluation example of the protein determination is described below.

First, 7 mL of the liquid A of PROTEIN ASSAY BCA KIT (Wako Pure Chemical Industries, Ltd.) and 140 μL of the liquid B thereof were mixed, and the prepared liquid was adopted as a liquid AB. Next, 25 μL (particle amount: 25 μg) of the dispersion (0.1% solution) of the affinity particles was taken, and was loaded into a 1.5 mL tube. Next, 200 μL of the liquid AB was added to the dispersion (25 μL), and the mixture was incubated at 60° C. for 30 minutes. The resultant solution was centrifuged at 4° C. and 15,000 rpm (20,400 g) for 5 minutes, and 200 μL of the supernatant was loaded into a 96-well microwell with a pipetter. The absorbance of the supernatant at 562 nm was measured with a microplate reader together with standard samples (several samples were obtained by diluting the antibody with 10 mM HEPES so that its concentration fell within the range of from 0 μg/mL to 200 μg/mL). The amount of the antibody was calculated from a standard curve. The amount of the antibody sensitized to the particles (the amount of the bonded antibody per weight of the particles (μg/mg)) was determined by dividing the calculated antibody amount by the weight of the particles (herein, 0.025 mg). Finally, the sensitization ratio was calculated. In the case where the antibody loading amount is 25 μg per 1 mg of the particles, when the antibody sensitization amount is 12.5 μg/mg, the sensitization ratio is 50%. The results are summarized in Table 1.
A: The sensitization ratio was 70% or more, and a sufficient amount of the antibody was sensitized.
B: The sensitization ratio was 30% or more and less than 70%, and a slight amount of the antibody was sensitized.
C: The sensitization ratio was less than 30%, and a sufficient amount of the antibody was not sensitized.

### (Evaluation 3: Evaluation of Latex Agglutination Sensitivity of Affinity Particles)

Standard serum for CRP was diluted with the PBS to a concentration of 0.75 mg/dL, and the resultant was used as a CRP specimen solution. A mixed liquid obtained by mixing 1 μL of the CRP specimen solution and 50 μL of a buffer (PBS containing 0.01% Tween 20) (hereinafter referred to as “R¹+”) was prepared and held at 37° C.

Next, 50 μL of each of the above-mentioned dispersions of affinity particles in which the affinity particles were sufficiently dispersed again with an ultrasonic wave before use (particle concentration: 0.1 mass %) was mixed with the R¹+. The absorbance at a wavelength of 572 nm of the mixed liquid (volume: 101 μL) immediately after stirring was measured. A spectrophotometer Biospectrometer manufactured by Eppendorf SE was used in the absorbance measurement. Then, the mixed liquid was left at rest at 37° C. for 5 minutes, and then its absorbance at a wavelength of 572 nm was measured again, followed by the calculation of the value of “(variation AABS in absorbance)×10,000.” The mixed liquid of each of the affinity particles and R¹+ was evaluated for the value of “(variation AABS in absorbance)×10,000” based on the following criteria, and the results are summarized in Table 1. Affinity particles having a larger value of “(variation AABS in absorbance)×10,000” of the R+ are expected to be capable of detecting a target substance with higher sensitivity when used as particles for the latex agglutination method in the specimen test.
AA: ΔABS×10,000 was 5,000 or more, and the sensitivity was extremely satisfactory.
A: ΔABS×10,000 was 1,000 or more and less than 5,000, and the sensitivity was satisfactory.
B: ΔABS×10,000 was 100 or more and less than 1,000, and the sensitivity was insufficient.
C: ΔABS×10,000 was less than 100, and almost no sensitivity was obtained.

TABLE 1

| | Example | Example | Example | Example | Example | Example | Comparative | Comparative |
|---|---|---|---|---|---|---|---|---|
| | 1 | 2 | 3 | 4 | 5 | 6 | Example 1 | Example 2 |
| | Particle | | | | | | | |
| | Particle 1 | Particle 2 | Particle 3 | Particle 4 | Particle 5 | Particle 6 | Particle 7 | Particle 8 |
| Evaluation 1 | AA | AA | AA | AA | AA | AA | A | C |
| Evaluation 2 | A | A | A | A | A | A | B | B |
| Evaluation 3 | A | A | A | A | A | A | B | C |

According to the present invention, there can be provided the support that has low nonspecific adsorption and is capable of detecting a target substance having a low concentration, the ligand-bonded support, and the affinity particle. There can be further provided the in vitro diagnostic reagent and the kit each including the particle, and the detection method.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2024-019747, filed Feb. 13, 2024, which is hereby incorporated by reference herein in its entirety.

What is claimed is:

1. A support comprising: a substrate; and a structure having a reactive functional group bonded to the substrate through a structure represented by the following formula (1),
wherein the structure having a reactive functional group has as the reactive functional group at least one selected from the group consisting of: a carboxy group; a maleimide group; a carbamoyl group; a tosyl group; an amino group; an epoxy group; and a thiol group: 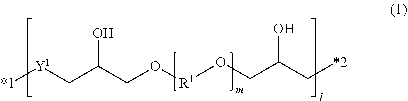
in the formula (1),
“l” represents an integer of 1 or more and 11 or less,
“m” represents an integer of 1 or more and 10 or less,
Y¹ represents a sulfur atom, a structure represented by the following formula (2), or a structure represented by the following formula (3),
R¹ represents an unsubstituted alkylene group having 2 to 4 carbon atoms,
1 represents a bonding position to a carbon atom or a sulfur atom in the substrate or the structure having a reactive functional group, and
2 represents a bonding position to a nitrogen atom, a sulfur atom, or an oxygen atom in the substrate or the structure having a reactive functional group,
provided that when *1 represents a bonding position to an atom in the substrate, *2 represents a bonding position to an atom in the structure having a reactive functional group, and when *1 represents a bonding position to an atom in the structure having a reactive functional group, *2 represents a bonding position to an atom in the substrate: 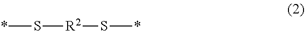
in the formula (2),
R² represents an alkylene group having 1 to 8 carbon atoms, and the alkylene group represented by R² may have at least one of a halogen atom and a hydroxy group as a substituent, and
* represents a bonding position in the structure represented by the formula (1); 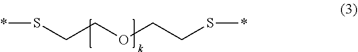
in the formula (3),
“k” represents an integer of 1 or more and 8 or less, and
* represents a bonding position in the structure represented by the formula (1).

2. The support according to claim 1, wherein the structure having a reactive functional group is at least one of a structure represented by the following formula (3-1), a structure represented by the following formula (3-2), a structure represented by the following formula (3-3), and a structure represented by the following formula (3-4): 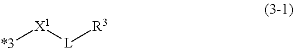
in the formula (3-1),
L represents an alkylene group having 1 to 6 carbon atoms, and the alkylene group represented by L may have at least one of a halogen atom and a hydroxy group as a substituent,
X¹ represents an imino group, a sulfur atom, or an oxygen atom,
*3 represents a bonding position to a carbon atom of the structure represented by the formula (1), and
R³ represents the reactive functional group; 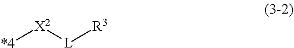
in the formula (3-2),
L represents an alkylene group having 1 to 6 carbon atoms, and the alkylene group represented by L may have at least one of a halogen atom and a hydroxy group as a substituent,
X² represents a methylene group or a sulfur atom,
*4 represents a bonding position to a sulfur atom of the structure represented by the formula (1), and
R³ represents the reactive functional group; 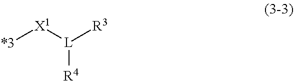
in the formula (3-3),
L represents a trivalent saturated hydrocarbon group having 1 to 6 carbon atoms, and the saturated hydrocarbon group represented by L may have at least one of a halogen atom and a hydroxy group as a substituent,
X¹ represents an imino group, a sulfur atom, or an oxygen atom,
*3 represents a bonding position to a carbon atom of the structure represented by the formula (1), and
R³ and R⁴ each represent the reactive functional group; 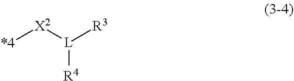
in the formula (3-4),
L represents a trivalent saturated hydrocarbon group having 1 to 6 carbon atoms, and the saturated hydrocarbon group represented by L may have at least one of a halogen atom and a hydroxy group as a substituent,
X² represents a methylene group or a sulfur atom,
*4 represents a bonding position to a sulfur atom of the structure represented by the formula (1), and
R³ and R⁴ each represent the reactive functional group.

3. The support according to claim 1, wherein the substrate contains a polymer having a structural unit derived from styrenes.

4. The support according to claim 1, wherein the substrate contains a polymer having a structural unit represented by the following formula (4-1): 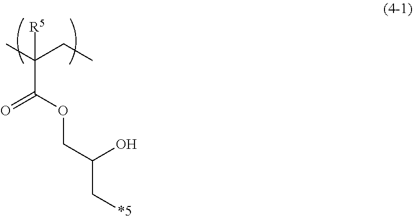
in the formula (4-1),
R⁵ represents a hydrogen atom or a methyl group, and
*5 represents a bonding position to a sulfur atom of the structure represented by the formula (1).

5. The support according to claim 1, wherein in the structure represented by the formula (1), R¹ represents an alkylene group having 2 carbon atoms.

6. The support according to claim 1, wherein in the structure represented by the formula (1), “m” represents an integer of 1 or more and 4 or less.

7. The support according to claim 1, wherein in the structure represented by the formula (1), Y¹ represents the structure represented by the formula (2), and in the structure represented by the formula (2), R² represents an alkylene group having 2 carbon atoms.

8. The support according to claim 1, wherein in the structure represented by the formula (1), “m” represents 1.

9. The support according to claim 1, wherein in the structure represented by the formula (1), “1” represents an integer of 1 or more and 6 or less.

10. The support according to claim 1, wherein the substrate has a particle shape.

11. A ligand-bonded support comprising: the support according to claim 1; and
a ligand chemically bonded to the support through the reactive functional group.

12. The ligand-bonded support according to claim 11, wherein the ligand is any one of an antigen, an antibody, an enzyme protein, a receptor of an enzyme protein, a signal substance, a receptor of a signal substance, and a nucleic acid.

13. An affinity particle comprising: the support of claim 10; and a ligand chemically bonded to the support through the reactive functional group.

14. A reagent for use in detection of a target substance in a specimen by in vitro diagnosis, the reagent comprising the affinity particle of claim 13.

15. A kit for use in detection of a target substance in a specimen by in vitro diagnosis, the kit comprising a cartridge including the affinity particle of claim 13.

16. A method of detecting a target substance in a specimen by in vitro diagnosis, the method comprising mixing the reagent of claim 14 and a specimen that may contain the target substance.

17. The method according to claim 16, further comprising measuring agglutination of the affinity particle by a change in absorbance to detect the presence or absence or a concentration of the target substance.

18. A compound represented by the following formula (5-1),

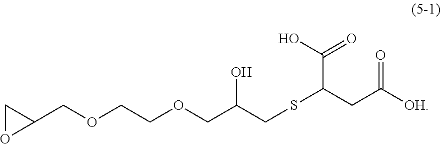 [SMILES: O=C(O)CC(SCC(O)COCCOCC1CO1)C(=O)O]

19. A compound represented by the following formula (5-2), 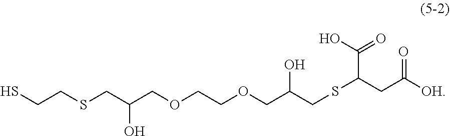 [SMILES: O=C(O)CC(SCC(O)COCCOCC(O)CSCCS)C(=O)O]
